(19) United States
(12) Patent Application Publication
(10) Pub. No.: US 2025/0264559 A1
(43) Pub. Date: Aug. 21, 2025

(54) LOW-FIELD MAGNETIC RESONANCE FLOW IMAGING

(71) Applicant: neuro42, Inc., San Francisco, CA (US)

(72) Inventors: Benjamin Inglis, Petaluma, CA (US); Lawrence Wald, San Francisco, CA (US); Nio Anderson, San Francisco, CA (US)

(73) Assignee: neuro42, Inc., San Francisco, CA (US)

(21) Appl. No.: 18/582,141

(22) Filed: Feb. 20, 2024

(51) Int. Cl. G01R 33/48; A61B 5/00; A61B 5/026; G01R 33/3873; G01R 33/50

(52) U.S. Cl. CPC G01R 33/4806; A61B 5/0042; A61B 5/0263; G01R 33/3873; G01R 33/50

(57) ABSTRACT

The present disclosure describes a device, system, and methods for low-field magnetic resonance (MR) imaging. For example, a method for low-field MR imaging can include projecting a primary static magnetic field in a field of view, projecting a secondary static magnetic field to pre-polarize a first fluid moving toward the field of view, and acquiring a first image of the first fluid in the field of view based on a first RF pulse sequence. The method can further include projecting a tertiary static magnetic field to pre-polarize a second fluid moving toward the field of view and acquiring a second image of the second fluid in the field of view based on a second RF pulse sequence. The first image can be subtracted from the second image to generate a flow image of an object of interest positioned in the field of view.

### BACKGROUND

The present disclosure relates to magnetic resonance imaging (MRI), medical imaging, medical intervention, and surgical intervention. MRI systems often include large and complex machines that generate significantly high magnetic fields and create significant constraints on the feasibility of certain surgical interventions. Restrictions can include limited physical access to the patient by a surgeon and/or a surgical robot and/or limitations on the usage of certain electrical and mechanical components in the vicinity of the MRI scanner. Such limitations are inherent in the underlying design of many existing systems and are difficult to overcome.

### SUMMARY

In one aspect, the present disclosure describes a method. The method can include projecting a primary static magnetic field in a field of view. The primary static magnetic field can include a low-field strength magnetic field. The field of view can be defined within a head-optimized housing for magnetic resonance (MR) imaging. The method can further include projecting a secondary static magnetic field to pre-polarize a first fluid outside the field of view and moving toward the field of view, transmitting a first radio frequency (RF) pulse sequence to an RF coil assembly to excite magnetization in the first fluid in the field of view, and acquiring a first image of the first fluid in the field of view based on the first RF pulse sequence. The method can further include projecting a tertiary static magnetic field to pre-polarize a second fluid outside the field of view and moving toward the field of view, transmitting a second RF pulse sequence to the RF coil assembly to excite magnetization in the second fluid in the field of view, and acquiring a second image of the second fluid in the field of view based on the second RF pulse sequence. The tertiary static magnetic field can be oriented opposite to the secondary static magnetic field. The first image can be subtracted from the second image to generate a flow image of an object of interest positioned in the field of view.

In another aspect, the present disclosure describes a magnetic resonance (MR) imaging system. The MR imaging system can include a domed housing, an array of magnets mounted to the domed housing, a secondary magnet, a radio frequency (RF) coil assembly, and a control circuit. The array of magnets can be configured to generate a static, low-field strength magnetic field in a field of view. The static, low-field strength magnetic field can define a B₀ axis. The RF coil assembly can be configured to excite magnetization in an object of interest positioned in the field of view. The secondary magnet can be separate from the domed housing and can be movable between a first configuration and a second configuration. The secondary magnet can be configured to generate a secondary static magnetic field. The secondary static magnetic field can define a secondary axis that is parallel to the B₀ axis in the first configuration of the secondary magnetic and is anti-parallel to the B₀ axis in the second configuration of the secondary magnet. The control circuit can transmit RF pulse sequences to the RF coil assembly, receive first output signals from the RF coil assembly corresponding to the secondary magnet being positioned in the first configuration, and receive second output signals from the RF coil assembly corresponding to the secondary magnet being positioned in the second configuration. The control circuit can generate an image based on the difference between the first output signals and the second output signals.

### BRIEF DESCRIPTION OF THE DRAWINGS

The various aspects described herein, both as to organization and methods of operation, together with further objects and advantages thereof, may best be understood by reference to the following description, taken in conjunction with the accompanying drawings as follows.

FIG. 1 depicts components of a MRI scanning system including a dome-shaped housing for a magnetic array, the dome-shaped housing surrounding a region of interest therein and further depicting the dome-shaped housing positioned to receive at least a portion of the head of a patient reclined on the table into the region of interest, in accordance with at least one aspect of the present disclosure.

FIG. 2 is a perspective view of an alternative dome-shaped housing for a magnetic array for use with the MRI scanning system of FIG. 1, wherein access apertures are defined in the dome-shaped housing, in accordance with at least one aspect of the present disclosure.

FIG. 3 is a perspective view of an alternative dome-shaped housing for a magnetic array for use with the MRI scanning system of FIG. 1, wherein access apertures and an adjustable gap is defined in the dome-shaped housing, in accordance with at least one aspect of the present disclosure.

FIG. 4 depicts a dome-shaped housing for use with a MRI scanning system having an access aperture in the form of a centrally-defined hole, in accordance with at least one aspect of the present disclosure.

FIG. 5 is a cross-sectional view of the dome-shaped housing of FIG. 4, in accordance with at least one aspect of the present disclosure.

FIG. 6 depicts a control schematic for a MRI system, in accordance with at least one aspect of the present disclosure.

FIG. 7 is a flowchart describing a method for obtaining imaging data from an MRI system, in accordance with at least one aspect of the present disclosure.

FIG. 8 depicts a MRI scanning system and a robotic system, in accordance with at least one aspect of the present disclosure.

FIG. 9 depicts portions of a head-optimized MRI system in which the main magnetic field B₀ is oriented orthogonal to the patient's body, the system including a pre-polarizing planar electromagnetic coil, in accordance with at least one aspect of the present disclosure.

FIG. 10 depicts portions of a head-optimized MRI system in which the main magnetic field B₀ is oriented orthogonal to the patient's body, the system including a pre-polarizing electromagnetic Helmholtz coil pair, in accordance with at least one aspect of the present disclosure.

FIG. 11 depicts portions of a head-optimized MRI system in which the main magnetic field B₀ is oriented axially along the patient's body, the system including a pre-polarizing electromagnetic planar coil, in accordance with at least one aspect of the present disclosure.

FIG. 12 depicts portions of a head-optimized MRI system in which the main magnetic field B₀ is oriented axially along the patient's body, the system including a pre-polarizing electromagnetic Helmholtz coil pair, in accordance with at least one aspect of the present disclosure.

FIG. 13 depicts portions of a head-optimized MRI system in which the main magnetic field B₀ is oriented axially along the patient's body, the system including a pre-polarizing electromagnetic solenoid coil, in accordance with at least one aspect of the present disclosure.

FIG. 14 depicts portions of a head-optimized MRI system in which the main magnetic field B₀ is oriented orthogonal to the patient's body, the system including a pre-polarizing permanent magnet, in accordance with at least one aspect of the present disclosure.

FIG. 15 depicts portions of a head-optimized MRI system in which the main magnetic field B₀ is oriented axially along the patient's body, the system including a pre-polarizing permanent magnet, in accordance with at least one aspect of the present disclosure.

FIG. 16 depicts pulse sequence diagrams for generating a flow image, in accordance with at least one aspect of the present disclosure.

FIG. 17 depicts a flow chart for generating a flow image using a secondary pre-polarizing magnet, in accordance with at least one aspect of the present disclosure.

FIGS. 18-20 graphically depict the relative field strength of a pre-polarizing magnetic field and a shim magnetic field for various head-optimized MRI systems, in accordance with at least one aspect of the present disclosure.

Corresponding reference characters indicate corresponding parts throughout the several views. The exemplifications set out herein illustrate various disclosed embodiments, is one form, and such exemplifications are not to be construed as limiting the scope thereof in any manner.

### DETAILED DESCRIPTION

Applicant of the present application also owns the following patent applications, which are each herein incorporated by reference in their respective entireties:
International Patent Application No. PCT/US2022/72143, titled NEURAL INTERVENTIONAL MAGNETIC RESONANCE IMAGING APPARATUS, filed May 5, 2022;
U.S. patent application Ser. No. 18/057,207, titled SYSTEM AND METHOD FOR REMOVING ELECTROMAGNETIC INTERFERENCE FROM LOW-FIELD MAGNETIC RESONANCE IMAGES, filed Nov. 19, 2022;
U.S. patent application Ser. No. 18/147,418, titled MODULARIZED MULTI-PURPOSE MAGNETIC RESONANCE PHANTOM, filed Dec. 28, 2022;
U.S. patent application Ser. No. 18/147,542, titled INTRACRANIAL RADIO FREQUENCY COIL FOR INTRAOPERATIVE MAGNETIC RESONANCE IMAGING, filed Dec. 28, 2022;
U.S. patent application Ser. No. 18/147,556, titled DEEP LEARNING SUPER-RESOLUTION TRAINING FOR ULTRA LOW-FIELD MAGNETIC RESONANCE IMAGING, filed Dec. 28, 2022;
U.S. patent application Ser. No. 18/153,111, titled ACCELERATING MAGNETIC RESONANCE IMAGING USING PARALLEL IMAGING AND ITERATIVE IMAGE RECONSTRUCTION, filed Jan. 11, 2023;
U.S. patent application Ser. No. 18/153,175, titled FAST T2-WEIGHTED AND DIFFUSION-WEIGHTED CHIRPED-CPMG SEQUENCES, filed Jan. 11, 2023;
U.S. patent application Ser. No. 18/450,010, titled ITERATIVE SHIMMING FOR LOW-FIELD HEAD-OPTIMIZED MRI, filed Aug. 15, 2023;
U.S. patent application Ser. No. 18/459,712, titled ACTIVE SHIMMING FOR LOW-FIELD MAGNETIC RESONANCE IMAGING, filed Sep. 1, 2023; and
U.S. patent application Ser. No. 18/407,758, titled DISTORTION CORRECTION OF LOW FIELD MAGNETIC RESONANCE IMAGES WITH PAIRED HIGH FIELD MAGNETIC RESONANCE IMAGES USING MACHINE LEARNING, filed Jan. 9, 2024.

Before explaining various aspects of interventional magnetic resonance imaging devices in detail, it should be noted that the illustrative examples are not limited in application or use to the details of construction and arrangement of parts illustrated in the accompanying drawings and description. The illustrative examples may be implemented or incorporated in other aspects, variations and modifications, and may be practiced or carried out in various ways. Further, unless otherwise indicated, the terms and expressions employed herein have been chosen for the purpose of describing the illustrative examples for the convenience of the reader and are not for the purpose of limitation thereof. Also, it will be appreciated that one or more of the following-described aspects, expressions of aspects, and/or examples, can be combined with any one or more of the other following-described aspects, expressions of aspects and/or examples.

Various aspects are directed to neural interventional magnetic resonance imaging (MRI) devices that allows for the integration of surgical intervention and guidance with an MRI. This includes granting physical access to the area around the patient as well as access to the patient's head with one or more access apertures. In addition, the neural interventional MRI device may allow for the usage of robotic guidance tools and/or traditional surgical implements. In various instances, a neural interventional MRI can be used intraoperatively to obtain scans of a patient's head and/or brain during a surgical intervention, such as a surgical procedure like a brain biopsy or neurosurgery.

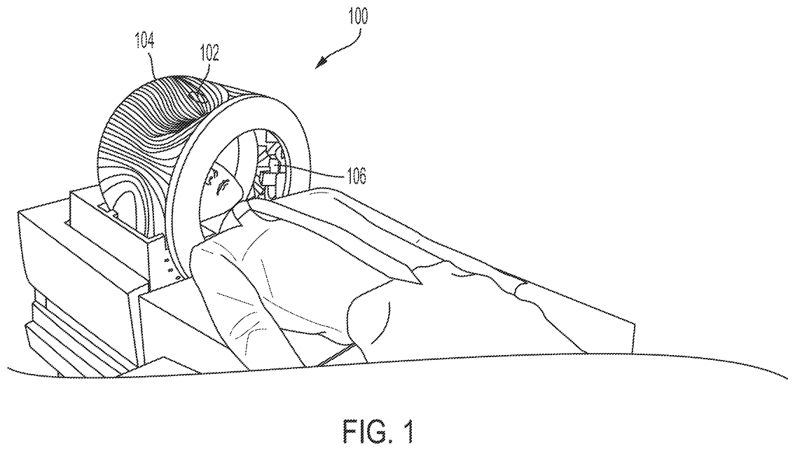

FIG. 1 depicts a MRI scanning system 100 that includes a dome-shaped housing 102 configured to receive a patient's head. The dome-shaped housing 102 can further include at least one access aperture configured to allow access to the patient's head to enable a neural intervention. A space within the dome-shaped housing 102 forms the region of interest for the MRI scanning system 100. Target tissue in the region of interest is subjected to magnetization fields/pulses, as further described herein, to obtain imaging data representative of the target tissue.

For example, a patient can be positioned such that his/her head is positioned within the region of interest within the dome-shaped housing 102. The brain can be positioned entirely within the dome-shaped housing 102. In such instances, to facilitate intracranial interventions (e.g. neurosurgery) in concert with MR imaging, the dome-shaped housing 102 can include one or more apertures that provide access to the brain. Apertures can be spaced apart around the perimeter of the dome-shaped housing.

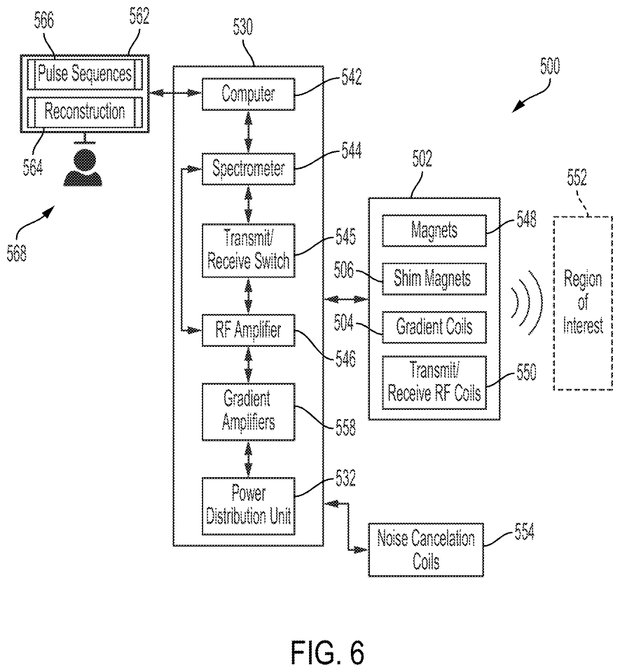

The MRI scanning system 100 can include an auxiliary cart (see, e.g. auxiliary cart 540 in FIG. 6) that houses certain conventional MRI electrical and electronic components, such as a computer, programmable logic controller, power distribution unit, and amplifiers, for example. The MRI scanning system 100 can also include a magnet cart that holds the dome-shaped housing 102, gradient coil(s), and/or a transmission coil, as further described herein. Additionally, the magnet cart can be attached to a receive coil in various instances. Referring primarily to FIG. 1, the dome-shaped housing 102 can further include RF transmission coils, gradient coils 104 (depicted on the exterior thereof), and shim magnets 106 (depicted on the interior thereof). Alternative configurations for the gradient coil(s) 104 and/or shim magnets 106 are also contemplated. In various instances, the shim magnets 106 can be adjustably positioned in a shim tray within the dome-shaped housing 102, which can allow a technician to granularly configure the magnetic flux density of the dome-shaped housing 102.

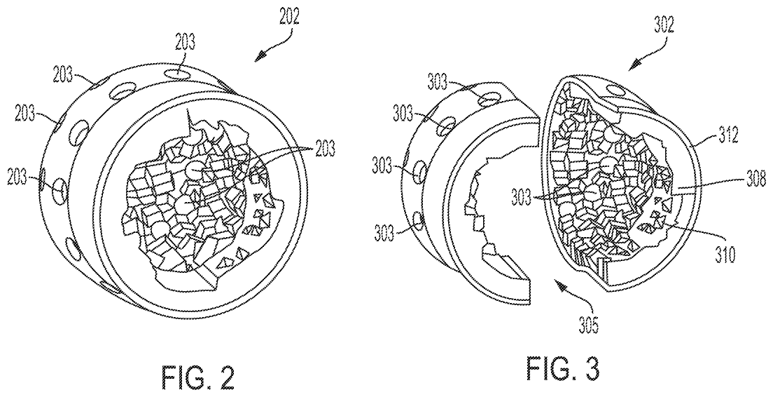

Various structural housings for receiving the patient's head and enabling neural interventions can be utilized with a MRI scanning system, such as the MRI scanning system 100. In one aspect, the MRI scanning system 100 may be outfitted with an alternative housing, such as a dome-shaped housing 202 (FIG. 2) or a two-part housing 302 (FIG. 3) configured to form a dome-shape. The dome-shaped housing 202 defines a plurality of access apertures 203; the two-part housing 302 also defines a plurality of access apertures 303 and further includes an adjustable gap 305 between the two parts of the housing.

In various instances, the housings 202 and 302 can include a bonding agent 308, such as an epoxy resin, for example, that holds a plurality of magnetic elements 310 in fixed positions. The plurality of magnetic elements 310 can be bonded to a structural housing 312, such as a plastic substrate, for example. In various aspects, the bonding agent 308 and structural housing 312 may be non-conductive or diamagnetic materials. Referring primarily to FIG. 3, the two-part housing 302 comprises two structural housings 312. In various aspect, a structural housing for receiving the patient's head can be formed from more than two sub-parts. The access apertures 303 in the structural housing 312 provide a passage directly to the patient's head and are not obstructed by the structural housing 312, bonding agent 308, or magnetic elements 310. The access apertures 303 can be positioned in an open space of the housing 302, for example.

There are many possible configurations of neural interventional MRI devices that can achieve improved access for surgical intervention. Many configurations build upon two main designs, commonly known as the Halbach cylinder and the Halbach dome described in the following article: Cooley et al. (e.g. Cooley, C. Z., Haskell, M. W., Cauley, S. F., Sappo, C., Lapierre, C. D., Ha, C. G., Stockmann, J. P., & Wald, L. L. (2018). Design of sparse Halbach magnet arrays for portable MRI using a genetic algorithm. IEEE transactions on magnetics, 54(1), 5100112. The article “Design of sparse Halbach magnet arrays for portable MRI using a genetic algorithm” by Cooley et al., published in IEEE transactions on magnetics, 54(1), 5100112 in 2018, is incorporated by reference herein in its entirety.

In various instances, a dome-shaped housing for an MRI scanning system, such as the system 100, for example, can include a Halbach dome defining a dome shape and configured based on several factors including main magnetic field B₀ strength, field size, field homogeneity, device size, device weight, and access to the patient for neural intervention. In various aspects, the Halbach dome comprises an exterior radius and interior radius at the base of the dome. The Halbach dome may comprise an elongated cylindrical portion that extends from the base of the dome. In one aspect, the elongated cylindrical portion comprises the same exterior radius and interior radius as the base of the dome and continues from the base of the dome at a predetermined length, at a constant radius. In another aspect, the elongated cylindrical portion comprises a different exterior radius and interior radius than the base of the dome (see e.g. FIGS. 2 and 3). In such instances, the different exterior radius and interior radius of the elongated cylindrical portion can merge with the base radii in a transitional region.

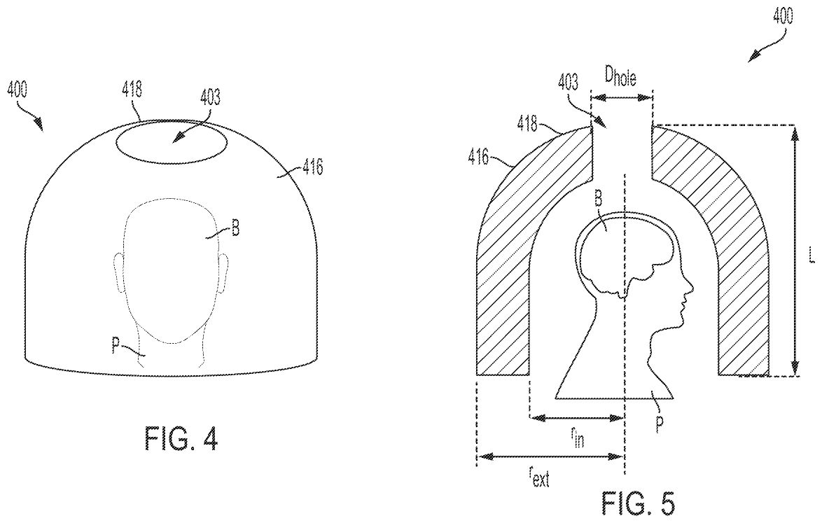

FIG. 4 illustrates an exemplary Halbach dome 400 for an MRI scanning system, such as the system 100, for example, which defines an access aperture in the form of a hole or access aperture 403, where the dome 400 is configured to receive a head and brain B of the patient P within the region of interest therein, and the access aperture 403 is configured to allow access to the patient P to enable neural intervention with a medical instrument and/or robotically-controlled surgical tool, in accordance with at least one aspect of the present disclosure. The Halbach dome 400 can be built with a single access aperture 403 at the top side 418 of the dome 400, which allows for access to the top of the skull while minimizing the impact to the magnetic field. Additionally or alternatively, the dome 300 can be configured with multiple access apertures around the structure 416 of the dome 400, as shown in FIGS. 2 and 3.

The diameter Dₕₒₗₑ of the access aperture 403 may be small (e.g. about 2.54 cm) or very large (substantially the exterior rₑₓₜ diameter of the dome 400). As the access aperture 403 becomes larger, the dome 400 begins to resemble a Halbach cylinder, for example. The access aperture 403 is not limited to being at the apex of the dome 400. The access aperture 403 can be placed anywhere on the surface or structure 416 of the dome 400. In various instances, the entire dome 400 can be rotated so that the access aperture 403 can be co-located with a desired physical location on the patient P.

FIG. 5 depicts relative dimensions of the Halbach dome 400, including a diameter Dₕₒₗₑ of the access aperture 403, a length L of the dome 400, and an exterior radius rₑₓₜ and an interior radius rᵢₙ of the dome 400. The Halbach dome 400 comprises a plurality of magnetic elements that are configured in a Halbach array and make up a magnetic assembly. The plurality of magnetic elements may be enclosed by the exterior radius rₑₓₜ and interior radius rᵢₙ in the structure 416 or housing thereof. In one aspect, example dimensions may be defined as: rᵢₙ=19.3 cm; rₑₓₜ=23.6 cm; L=38.7 cm; and 2.54 cm≤D<19.3 cm.

Based on the above example dimensions, a Halbach dome 400 with an access aperture 403 may be configured with a magnetic flux density B₀ of around 72 mT, and an overall mass of around 35 kg. It will be appreciated that the dimensions may be selected based on particular applications to achieve a desired magnetic flux density B₀, total weight of the Halbach dome 400 and/or magnet cart, and geometry of the neural intervention access aperture 403.

In various aspects, the Halbach dome 400 may be configured to define multiple access apertures 403 placed around the structure 416 of the dome 400. These multiple access apertures 403 may be configured to allow for access to the patient's head and brain B using tools (e.g., surgical tools) and/or a surgical robot.

In various aspects, the access aperture 403 may be adjustable. The adjustable configuration may provide the ability for the access aperture 403 to be adjusted using either a motor, mechanical assist, or a hand powered system with a mechanical iris configuration, for example, to adjust the diameter Dₕₒₗₑ of the access aperture 403. This would allow for configuration of the dome without an access aperture 403, conducting an imaging scan, and then adjusting the configuration of the dome 400 and mechanical iris thereof to include the access aperture 403 and, thus, to enable a surgical intervention therethrough.

Halbach domes and magnetic arrays thereof for facilitating neural interventions are further described in International Patent Application No. PCT/US2022/72143, titled NEURAL INTERVENTIONAL MAGNETIC RESONANCE IMAGING APPARATUS, filed May 5, 2022, which is incorporated by reference herein in its entirety.

Referring now to FIG. 6, a schematic for an MRI system 500 is shown. The MRI scanning system 100 (FIG. 1) and the various dome-shaped housings and magnetic arrays therefor, which are further described herein, for example, can be incorporated into the MRI system 500, for example. The MRI system 500 includes a housing 502, which can be similar in many aspects to the dome-shaped housings 102 (FIG. 1), 202 (FIG. 2), and/or 302 (FIG. 3), for example. The housing 502 is dome-shaped and configured to form a region of interest, or field of view, 552 therein. For example, the housing 502 can be configured to receive a patient's head in various aspects of the present disclosure.

The housing 502 includes a magnet assembly 548 having a plurality of magnets arranged therein (e.g. a Halbach array of magnets). In various aspect, the main magnetic field B₀, generated by the magnetic assembly 548, extends into the field of view 552, which contains an object (e.g. the head of a patient) that is being imaged by the MRI system 500.

The MRI system 500 also includes RF transmit/receive coils 550. The RF transmit/receive coils 550 are combined into integrated transmission-reception (Tx/Rx) coils. In other instances, the RF transmission coil can be separate from the RF reception coil. For example, the RF transmission coil(s) can be incorporated into the housing 502 and the RF reception coil(s) can be positioned within the housing 502 to obtain imaging data.

The housing 502 also includes one or more gradient coils 504, which are configured to generate gradient fields to facilitate imaging of the object in the field of view 552 generated by the magnet assembly 548, e.g., enclosed by the dome-shaped housing and dome-shaped array of magnetic elements therein. Shim trays adapted to receive shim magnets 506 can also be incorporated into the housing 502.

During the imaging process, the main magnetic field B₀ extends into the field of view 552. The direction of the effective magnetic field (B₁) changes in response to the RF pulses and associated electromagnetic fields transmitted by the RF transmit/receive coils 550. For example, the RF transmit/receive coils 550 may be configured to selectively transmit RF signals or pulses to an object in the field of view 552, e.g. tissue of a patient's brain. These RF pulses may alter the effective magnetic field experienced by the spins in the sample tissue.

The housing 502 is in signal communication with an auxiliary cart 530, which is configured to provide power to the housing 502 and send/receive control signals to/from the housing 502. The auxiliary cart 530 includes a power distribution unit 532, a computer 542, a spectrometer 544, a transmit/receive switch 545, an RF amplifier 546, and gradient amplifiers 558. In various instances, the housing 502 can be in signal communication with multiple auxiliary carts and each cart can support one or more of the power distribution unit 532, the computer 542, the spectrometer 544, the transmit/receive switch 545, the RF amplifier 546, and/or the gradient amplifiers 558.

The computer 542 is in signal communication with a spectrometer 544 and is configured to send and receive signals between the computer 542 and the spectrometer 544. When the object in the field of view 552 is excited with RF pulses from the RF transmit/receive coils 550, the precession of the object results in an induced electric current, or MR current, which is detected by the RF transmit/receive coils 550 and sent to the RF preamplifier 556. The RF preamplifier 556 is configured to boost or amplify the excitation data signals and send them to the spectrometer 544. The spectrometer 544 is configured to send the excitation data to the computer 542 for storage, analysis, and image construction. The computer 542 is configured to combine multiple stored excitation data signals to create an image, for example. In various instances, the computer 542 is in signal communication with at least one database 562 that stores reconstruction algorithms 564 and/or pulse sequences 566. The computer 542 is configured to utilize the reconstruction algorithms to generate an MR image 568.

From the spectrometer 544, signals can also be relayed to the RF transmit/receive coils 550 in the housing 502 via an RF power amplifier 546 and the transmit/receive switch 545 positioned between the spectrometer 544 and the RF power amplifier 546. From the spectrometer 544, signals can also be relayed to the gradient coils 560 in the housing 502 via a gradient power amplifier 558. For example, the RF power amplifier 546 is configured to amplify the signal and send it to RF transmission coils 560, and the gradient power amplifier 558 is configured to amplify the gradient coil signal and send it to the gradient coils 560.

In various instances, the MRI system 500 can include noise cancellation coils 554. For example, the auxiliary cart 530 and/or computer 542 can be in signal communication with noise cancellation coils 554. In other instances, the noise cancellation coils 554 can be optional. For example, certain MRI systems disclosed herein may not include supplemental/auxiliary RF coils for detecting and canceling electromagnetic interference, i.e. noise.

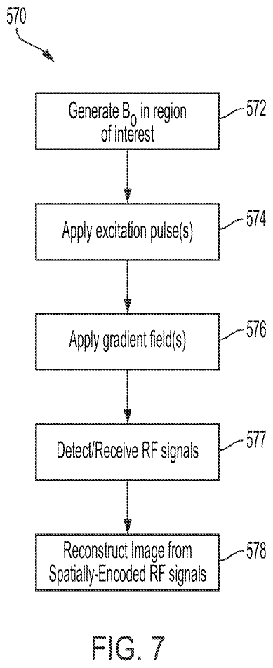

A flowchart depicting a process 570 for obtaining an MRI image is shown in FIG. 7. The flowchart can be implemented by the MRI system 500, for example. In various instances, at block 572, the target subject (e.g. a portion of a patient's anatomy), is positioned in a main magnetic field B₀ in an interest of region (e.g. region of interest 552), such as within the dome-shaped housing of the various MRI scanners further described herein (e.g. magnet assembly 548). The main magnetic field B₀ is configured to magnetically polarize the hydrogen protons (1H-protons) of the target subject (e.g. all organs and tissues) and is known as the net longitudinal magnetization Mo. It is proportional to the proton density (PD) of the tissue and develops exponentially in time with a time constant known as the longitudinal relaxation time T1 of the tissue. T1 values of individual tissues depend on a number of factors including their microscopic structure, on the water and/or lipid content, and the strength of the polarizing magnetic field, for example. For these reasons, the T1 value of a given tissue sample is dependent on age and state of health.

At block 574, a time varying oscillatory magnetic field B₁, i.e. an excitation pulse, is applied to the magnetically polarized target subject with a RF coil (e.g. RF transmit/receive coil 550). The carrier frequency of the pulsed B₁ field is set to the resonance frequency of the 1H-proton, which causes the longitudinal magnetization to flip away from its equilibrium longitudinal direction resulting in a rotated magnetization vector, which in general can have transverse as well as longitudinal magnetization components, depending on the flip angle used. Common B₁ pulses include an inversion pulse, or a 180-degree pulse, and a 90-degree pulse. A 180-degree pulse reverses the direction of the 1H-proton's magnetization in the longitudinal axis. A 90-degree pulse rotates the 1H-proton's magnetization by 90 degrees so that the magnetization is in the transverse plane. The MR signals are proportional to the transverse components of the magnetization and are time varying electrical currents that are detected with suitable RF coils. These MR signals decay exponentially in time with a time constant known as the transverse relaxation time T2, which is also dependent on the microscopic tissue structure, water/lipid content, and the strength of the magnetic field used, for example.

At block 576, the MR signals are spatially encoded by exposing the target subject to additional magnetic fields generated by gradient coils (e.g. gradient coils 560), which are known as the gradient fields. The gradient fields, which vary linearly in space, are applied for short periods of time in pulsed form and with spatial variations in each direction. The net result is the generation of a plurality of spatially encoded MR signals, which are detected at block 577, and which can be reconstructed to form MR images depicting slices of the examination subject. A RF reception coil (e.g. RF transmit/receive coil 550) can be configured to detect the spatially-encoded RF signals. Slices may be oriented in the transverse, sagittal, coronal, or any oblique plane.

At block 578, the spatially encoded signals of each slice of the scanned region are digitized and spatially decoded mathematically with a computer reconstruction program (e.g. by computer 542) in order to generate images depicting the internal anatomy of the examination subject. In various instances, the reconstruction program can utilize an (inverse) Fourier transform to back-transforms the spatially-encoded data (k-space data) into geometrically decoded data.

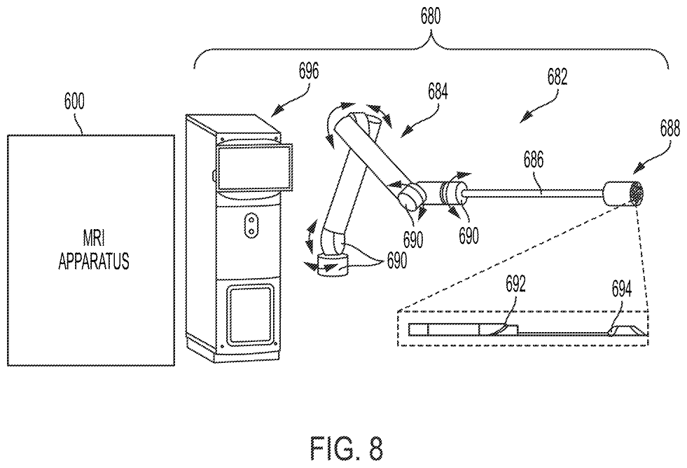

FIG. 8 depicts a graphical illustration of a robotic system 680 that may be used for neural intervention with an MRI scanning system 600. The robotic system 680 includes a computer system 696 and a surgical robot 682. The MRI scanning system 600 can be similar to the MRI system 500 and can include the dome-shaped housing and magnetic arrays having access apertures, as further described herein. For example, the MRI system 500 can include one or more access apertures defined in a Halbach array of magnets in the permanent magnet assembly to provide access to one or more anatomical parts of a patient being imaged during a medical procedure. In various instances, a robotic arm and/or tool of the surgical robot 682 is configured to extend through an access aperture in the permanent magnet assembly to reach a patient or target site. Each access aperture can provide access to the patient and/or surgical site. For example, in instances of multiple access apertures, the multiple access apertures can allow access from different directions and/or proximal locations.

In accordance with various embodiments, the robotic system 680 is configured to be placed outside the MRI system 600. As shown in FIG. 8, the robotic system 680 can include a robotic arm 684 that is configured for movements with one or more degrees of freedom. In accordance with various embodiments, the robotic arm 684 includes one or more mechanical arm portions, including a hollow shaft 686 and an end effector 688. The hollow shaft 686 and end effector 688 are configured to be moved, rotated, and/or swiveled through various ranges of motion via one or more motion controllers 690. The double-headed curved arrows in FIG. 8 signify exemplary rotational motions produced by the motion controllers 690 at the various joints in the robotic arm 684.

In accordance with various embodiments, the robotic arm 684 of the robotic system 682 is configured for accessing various anatomical parts of interest through or around the MRI scanning system 600. In accordance with various embodiments, the access aperture is designed to account for the size of the robotic arm 684. For example, the access aperture defines a circumference that is configured to accommodate the robotic arm 684, the hollow shaft 686, and the end effector 688 therethrough. In various instances, the robotic arm 684 is configured for accessing various anatomical parts of the patient from around a side of the magnetic imaging apparatus 600. The hollow shaft 686 and/or end effector 688 can be adapted to receive a robotic tool 692, such as a biopsy needle having a cutting edge 694 for collecting a biopsy sample from a patient, for example.

The reader will appreciate that the robotic system 682 can be used in combination with various dome-shaped and/or cylindrical magnetic housings further described herein. Moreover, the robotic system 682 and robotic tool 692 in FIG. 8 are exemplary. Alternative robotic systems can be utilized in connection with the various MRI systems disclosed herein. Moreover, handheld surgical instruments and/or additional imaging devices (e.g. an endoscope) and/or systems can also be utilized in connection with the various MRI systems disclosed herein.

In various aspects of the present disclosure, the MRI systems described herein can comprise low field MRI (LF-MRI) systems. In such instances, the main magnetic field B₀ generated by the permanent magnet assembly can be between 0.1 T and 1.0 T, for example. In other instances, the MRI systems described herein can comprise ultra-low field MRI (ULF-MRI) systems. In such instances, the main magnetic field B₀ generated by the permanent magnet assembly can be between 0.03 T and 0.1 T, for example.

Higher magnetic fields, such as magnetic fields above 1.0 T, for example, can preclude the use of certain electrical and mechanical components in the vicinity of the MRI scanner. For example, the existence of surgical instruments and/or surgical robot components comprising metal, specially ferrous metals, can be dangerous in the vicinity of higher magnetic fields because such tools can be drawn toward the source of magnetization. Moreover, higher magnetic fields often require specifically-designed rooms with additional precautions and shielding to limit magnetic interference. Despite the limitations on high field MRI systems, low field and ultra-low field MRI systems present various challenges to the acquisition of high quality images with sufficient resolution for achieving the desired imaging objectives.

LF- and ULF-MRI systems generally define an overall magnetic field homogeneity that is relatively poor in comparison to higher field MRI systems. For example, a dome-shaped housing for an array of magnets, as further described herein, can comprise a Halbach array of permanent magnets, which generate a magnetic field B₀ having a homogeneity between 1,000 ppm and 10,000 ppm in the region of interest in various aspects of the present disclosure.

Flow imaging with a head-only, low field MRI presents numerous challenges given the scope of the magnetic field and the relatively poor homogeneity thereof.

MR imaging of vascular structures, such as arteries and veins, for example, can provide helpful diagnostic information for clinicians. In certain instances, vascular imaging with MRI involves the use of endogenous and/or exogenous contrast. For example, strong vascular contrast can be obtained using exogenous contrast agents, such as gadolinium chelates. However, these techniques are less desirable in certain circumstances due to concerns related to invasiveness, nephrotic toxicity, and repeatability with exogenous contrasts. Alternatively, imaging techniques that rely on endogenous phenomena, such as flow velocity, for example, can minimize certain risks and/or concerns. For example, MR angiography (MRA) can be obtained using time-of-flight (TOF) or phase contrast (PC) flow imaging. Both TOF and PC flow imaging exploit the intrinsic motional differences between flowing blood and stationary brain tissues.

In the context of low-field MRI systems, such as the head-optimized, low-field MRI systems disclosed herein, the main magnetic field generally exhibits relatively poor homogeneity (in comparison to high-field MRIs). For example, variations of hundreds or even thousands of parts per million are common. Due to the inhomogeneity, MR angiography is very challenging. For example, PC methods are incompatible with poor magnetic field homogeneity because the dephasing from blood flowing through the heterogeneous main magnetic field will dominate the much smaller phase evolution from blood flow through a user-defined pulsed gradient. TOF imaging is also incompatible with poor magnetic field homogeneity. TOF imaging requires a series of thin slice acquisitions (for 2-D TOF imaging) or a small number of slabs (for 3-D TOF imaging), which require good magnetic field homogeneity for accurate spatial selectivity of RF pulses. Furthermore, there is a smaller longitudinal relaxation time constant (T₁) difference between blood and brain tissues at low field than at high field, reducing the apparent T₁ shortening effect produced by flowing blood in TOF methods.

Another challenge to flow imaging with a head-only scanner is the limited polarization of arterial blood. The relaxation time constant T₁ for blood is at least 450 ms in a magnetic field of 80 millitesla (mT) as described in Inglis B, Buckenmaier K, Sangiorgio P, Pedersen A F, Nichols M A, Clarke J. MRI Of The Human Brain at 130 Microtesla. Proc Natl Acad Sci USA. 2013 Nov. 26; 110(48):19194-201, for example. Moreover, the time for blood to travel from the heart to the head may be just 1-2 seconds. In such instances, a head-only polarizing magnet with limited magnetic field beyond the lower neck will only polarize a small fraction of the arterial blood until that blood has reached deep into the brain. At that point, it may be infeasible to produce an angiogram.

A system and method for producing higher polarization of arterial blood and simultaneously generating an on/off pattern of flow tagging can overcome various challenges outlined herein.

Angiography around stents is a challenge for TOF and PC methods at conventional magnetic field strengths because of the static dephasing caused by the magnetic susceptibility gradients around the implant. Such dephasing is somewhat analogous to the inherently poor magnetic field heterogeneity in low field MRI scanners. Arterial spin labeling (ASL) to generate blood contrast and to interrogate the blood magnetization using a short or zero TE scan has been used to address the static dephasing around stents. For example, a spatial ASL tag can be used to prepare the arterial (or venous) blood, where the static signal is canceled by subtracting a control scan with no tag from a scan with the tagged blood. The foregoing criteria can be satisfied on conventional whole body MRI systems having a whole body transmit coil because the heart is within the scanner.

In contrast to a whole body MRI system, at low field and with a head-only MRI system, accurate spatial labeling of arterial blood is challenging. For example, spatial labeling using either slab-selective inversion or a pseudo-continuous labeling plane in the neck are both likely to perform poorly due to the magnetic field heterogeneity. In the former case, the slab profile will not be rectangular, while in the latter case the inversion efficiency is degraded. Velocity-selective ASL preparation would also be confounded by magnetic field heterogeneity in the same way as PC-MRA methods, i.e. the inherent poor heterogeneity would compete with or dominate the pulsed linear gradients used to encode flow velocity.

At high field with whole-body polarizing magnets, one approach to labeling of blood is to use a separate radiofrequency (RF) coil for labeling and a different RF coil for imaging. The labeling coil is usually positioned over the neck and the combined effect of the arterial blood flow and RF field from the labeling coil is to invert the arterial blood magnetization, using flow-driven adiabatic inversion. Flow-driven inversion with a dedicated labeling RF coil in animals is described in the following references, for example:
Silva A C, Zhang W, Williams D S, Koretsky A P. Multi-Slice MRI Of Rat Brain Perfusion During Amphetamine Stimulation Using Arterial Spin Labeling. Magn Reson Med. 1995 February; 33 (2): 209-14;
Zhang W, Silva A C, Williams D S, Koretsky A P. NMR Measurement Of Perfusion Using Arterial Spin Labeling Without Saturation Of Macromolecular Spins. Magn Reson Med. 1995 March; 33 (3): 370-6; and
Zhang X, Nagaoka T, Auerbach E J, Champion R, Zhou L, Hu X, Duong T Q. Quantitative Basal CBF And CBF Fmri Of Rhesus Monkeys Using Three-Coil Continuous Arterial Spin Labeling. Neuroimage. 2007 Feb. 1; 34(3):1074-83.

Flow-driven inversion with a dedicated labeling RF coil in humans is described in the following references, for example:
Dixon W T, Du L N, Faul D D, Gado M, Rossnick S. Projection Angiograms Of Blood Labeled By Adiabatic Fast Passage. Magn Reson Med. 1986 June; 3 (3): 454-62;
Zaharchuk G, Ledden P J, Kwong K K, Reese T G, Rosen B R, Wald L L. Multislice Perfusion And Perfusion Territory Imaging In Humans With Separate Label And Image Coils. Magn Reson Med. 1999 June; 41 (6): 1093-8; and
Talagala S L, Ye F Q, Ledden P J, Chesnick S. Whole-Brain 3D Perfusion MRI At 3.0 T Using CASL With A Separate Labeling Coil. Magn Reson Med. 2004 July; 52 (1): 131-40.

In some cases, a labeling coil over the neck is insufficient to produce the desired inversion. For example, in mice, it was found that the small distance between the neck and brain necessitated a slightly different approach, with blood labeling being done with a dedicated RF coil placed over the animal's heart as described in, for example Muir E R, Shen Q, Duong T Q. Cerebral Blood Flow MRI In Mice Using The Cardiac-Spin-Labeling Technique. Magn Reson Med. 2008 September; 60 (3): 744-8.

U.S. Pat. No. 8,570,035 to Wemmer et al., titled MAGNETIC RESONANCE IMAGING OF LIVING SYSTEMS BY REMOTE DETECTION, suggested an alternate approach to flow imaging in which the signal-to-noise ratio (SNR) is boosted via remote detection with a highly sensitive, small detector distal to the encoding location. For example, the region to be examined (e.g. a blood vessel in the brain) can be prepared using a combination of RF and gradient pulses in a polarizing magnetic field and then, after the tagged blood has flowed away from the preparation region, it is detected with high sensitivity in a downstream location compatible with the location of a small sensor, such as at the jugular vein of the neck.

Unlike a full body scanner, the blood in the heart, some of which is destined for the patient's brain, is initially outside of the polarizing magnetic field in a head-only MRI scanner. A secondary magnet could be used to pre-polarize the arterial blood outside the head, prior to its flow into the brain. However, based on the blood pressure wave from the aorta to the circle of Willis, blood may take 1-3 seconds to reach the patient's brain, causing a significant decay of the pre-polarization of the arterial blood due to longitudinal relaxation. Therefore, it may be desirable to polarize the blood flowing into the brain as close to the brain as possible without undesirable interference arising between the primary magnet array and the secondary magnet(s), while simultaneously minimizing the time between pre-polarization and flow into the brain to maximize sensitivity. The magnet array in a dome-shaped housing of a low-field, head-optimized MRI can be carefully engineered for imaging and interference with such magnetic fields can significantly impact the image quality, for example. Still, the secondary magnet(s) should be close enough to the main magnetic field to prevent undue relaxation of the polarization between the secondary magnet(s) and the primary magnet array. Generally, a loss of at least 50% is expected before the blood reaches the region of interest. For example, the polarization can dissipate by 60%-80% before it reaches the brain.

To overcome the foregoing challenges in the context of low-field MRIs and/or head-optimized MRIs, flow imaging can be obtained by combining spatial blood labeling with a two-step measurement technique to produce an angiographic or cerebral blood flow (CBF) image. Stated differently, difficulties associated with low arterial blood polarization and on/off blood tagging control can be addressed using a secondary, pre-polarizing magnet placed over the upper body of the patient, for example, to produce user-controllable polarization in arterial blood that is destined to flow to the subject's brain. In such instances, the patient is subjected to two B₀ fields—a primary, main magnetic field projected toward fluid pathways of the target organ in the region of interest and a second, pre-polarizing magnetic field projected upstream of the fluid pathways in the region of interest. A series of images are obtained. For example, images can be obtained with the upstream arterial magnetization alternately inverted, e.g. the secondary magnetic field is either parallel or anti-parallel relative to the main magnetic field, and the difference between the two images can produce the flow image. For surgical access, the pre-polarizing magnet (Bₚ) can be removed during routine anatomical scanning using the head-only imaging system. Then, when needed, the pre-polarizing magnet can be positioned over the patient's upper torso and/or chest, for MRA and/or CBF imaging, for example. Upon completion of the vascular imaging session, the pre-polarizing magnet(s) can be again removed.

Images of vascular structures are often an important diagnostic tool. For example, flow imaging can be used to visualize stenosis or occlusions and/or to locate major blood vessels during an interventional procedure, such as tumor resection, for example. Moreover, intraoperative flow imaging using a low-field and/or head-only MRI, can be used for surgical guidance to assist clinicians during surgical interventions.

The flow imaging techniques further described herein include a secondary magnet, or second magnetic array, to pre-polarize the blood destined to flow into the region of interest. The polarization of the secondary magnet can be controlled to reverse the polarization of the blood flowing into the region of interest.

Pre-polarizing magnet(s) for flow imaging can assume different forms. In one instance, the pre-polarizing magnet is a loop electromagnetic coil. In other instances, more complicated electromagnetic coil designs can be employed. For example, the pre-polarizing magnet can comprise a Helmholtz pair, a solenoid or a saddle coil. In still other instances, a permanent magnet can be used. The pre-polarizing magnet(s) can be selected based on engineering constraints, such as the direction of B₀ for the MR scanner's main magnetic field.

The pre-polarizing magnet for use with a head-only MRI can be positioned over, adjacent to, and/or against the subject's chest, for example. The pre-polarizing magnet can be positioned outside the housing of a head-only MRI. In various instances, the positioning of the pre-polarizing magnetic can be selected based on balancing the flow rate, effective magnetic field, and relaxation time T₁ to ensure the polarization does not entirely dissipate before reaching the region of interest, as further described herein.

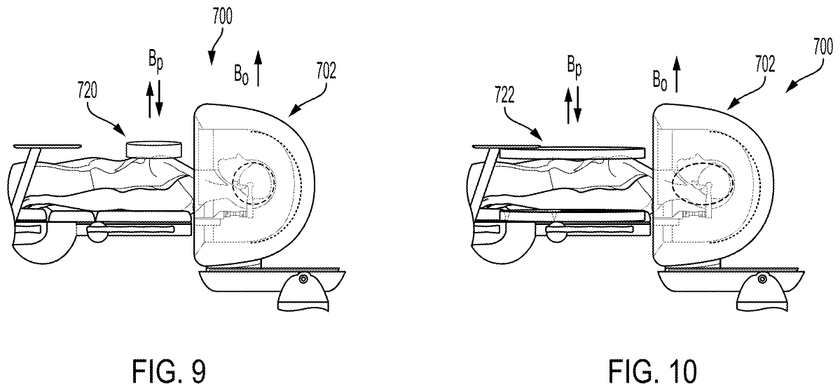

Referring now to FIGS. 9-15, portions of various MRI systems are depicted. These MRI systems are head-optimized MRI systems, which include the various components of the MRI system 500, including a domed housing with magnetic arrays (e.g. a Halbach array of magnets) supported therein, as further described herein. The domed housing and magnets thereof are positioned to form a region of interest, or field of view, within the domed housing for imaging a patient's head positioned therein. In various instances, the MRI systems of FIGS. 9-15 are further configured to utilize the process 570 (FIG. 7) to generate an image. Furthermore, the MRI systems of FIGS. 9-15 can be incorporated into a robotic system, such as the robotic system 680, for example, in certain instances.

FIGS. 9-10 depict a head-only MRI system 700 including a domed housing 702 generating a main magnetic field B₀ that is oriented in the anterior-posterior direction. The head-only MRI system 700 can be used with a pre-polarizing planar electromagnetic coil 720 (FIG. 9), or a pre-polarizing Helmholtz coil pair 722 (FIG. 10), to produce magnetization in arterial blood on a trajectory toward the brain, for example. The current direction in the pre-polarizing electromagnet 720, and 722 determines the polarity of the blood magnetization.

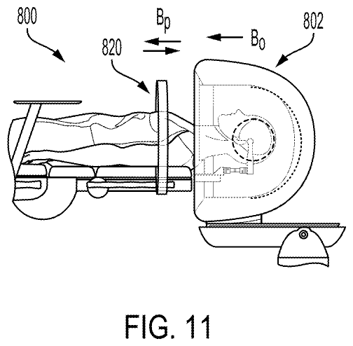

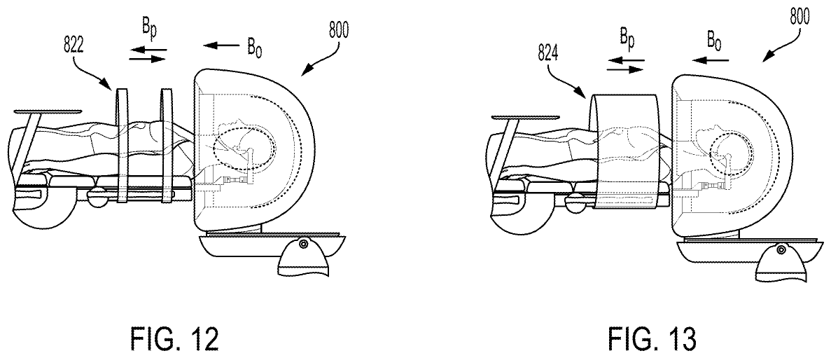

FIGS. 11-13 depict a head-only MRI system 800 including a domed housing 802 generating a main magnetic field B₀ that is oriented in the head-to-foot direction. The head-only MRI system 800 can be used with a pre-polarizing loop electromagnetic coil 820 (FIG. 11), a pre-polarizing Helmholtz coil pair 822 (FIG. 12), or a pre-polarizing solenoid coil 724 (FIG. 13) to produce magnetization in arterial blood destined for the brain, for example. The current direction in the pre-polarizing electromagnet 820, 822, and 824 determines the polarity of the blood magnetization.

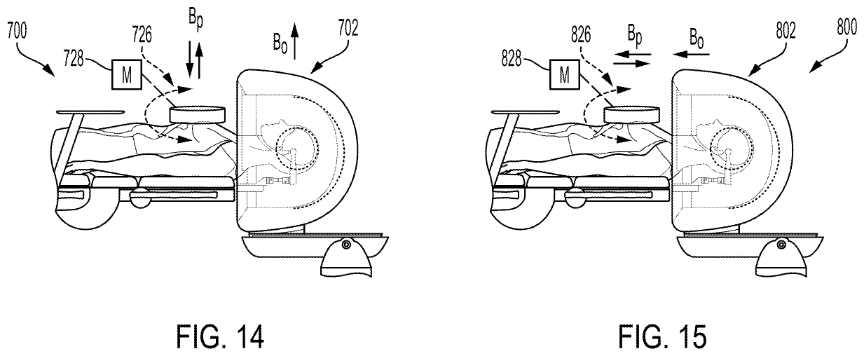

In various instances, a permanent magnet or permanent magnetic array can be used instead of an electromagnetic coil to pre-polarize the patient's blood for tagging. For example, FIG. 14 depicts the head-only MRI system 700 including a permanent magnet 726, which determines the polarity of the blood magnetization. FIG. 15 depicts the head-only MRI system 800 including a permanent magnet 826, which determines the polarity of the blood magnetization. The magnets 726, 826 can be reversed mechanically, e.g. rotated, to flip the polarity. The magnet(s) can be rotated to be either parallel or antiparallel to the main magnetic field B₀. In various instances, the magnets can be manually rotated. In still other instances, the magnets 726, 826 can be rotated by a motor 728, 828, respectively. For example, the magnets 726, 826 can be mounted to a frame, which is selectively rotatable based on the step in the imaging process, as further described herein.

In various instances, the secondary magnet(s) generate a tapered magnetic field. For example, the magnetic field can be teardrop-shaped. The magnetic field can correspond to a larger field over the patient's heart, for example, to maintain the magnetization and continue to grow the magnetization with a tapered field toward the region of interest. In various instances, a tapered magnetic field is configured to maintain the trajectory of the polarization as the arterial blood approaches the imaging region.

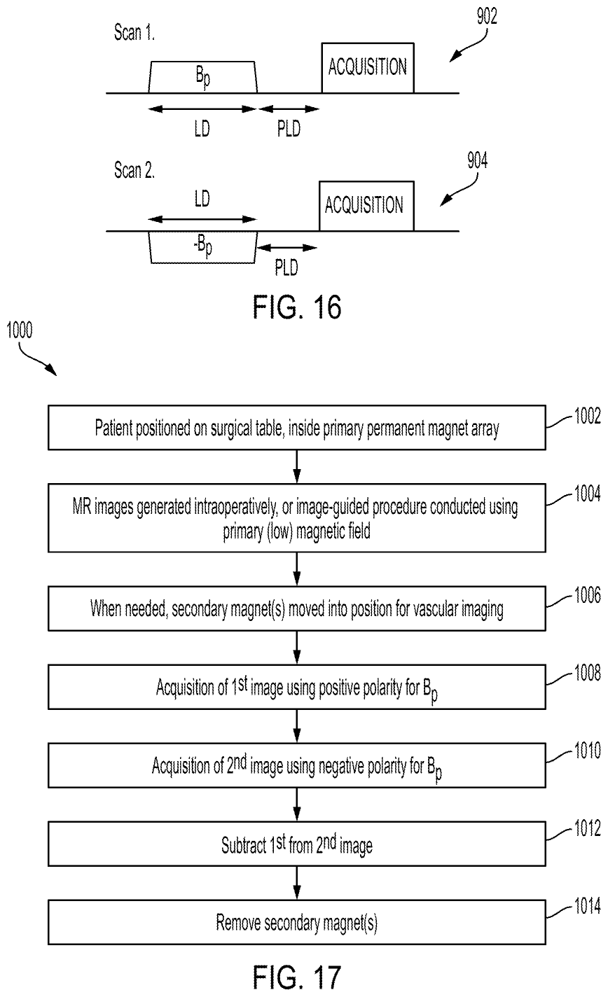

To generate the final MR angiography (MRA) or cerebral blood flow (CBF) image, the stationary MR signals in the brain are canceled from the image by subtraction. To do this, two images are recorded with opposite polarity magnetization for arterial blood, and one image is subtracted from the other. Referring primarily to FIG. 16, pulse sequence diagrams 902 and 904 are depicted. The first, or initial, pulse sequence diagram 902 includes a pre-polarizing labeling delay (LD), during which a first polarizing magnetization (Bₚ) is applied, followed by a post-labeling delay (PLD), before acquisition of the first MR image utilizing one of the various head-only scanners described herein. The second, or subsequent, pulse sequence diagram 904 includes a pre-polarizing labeling delay (LD), during which a second polarizing magnetization (−Bₚ) is applied, followed by a post-labeling delay (PLD), before acquisition of the second MR image utilizing one of the various head-only scanners further described herein. The second polarizing magnetization (−Bₚ) is antiparallel to the first polarizing magnetization (Bₚ). In various instances, the labeling delay (LD) time period corresponds to at least the relaxation time T1. In certain instances, the labeling delay (LD) time period is at least twice or thrice the relaxation time T1. For example, the labeling delay can be about twice the relaxation time T1, thrice the relaxation time T1, or between twice and thrice the relaxation time T1. In various instances, the post-labeling delay (PLD) time period is less than five times the relaxation time T1. In certain instances, the post-labeling delay (PLD) time period is about equal to the relaxation time T1, about twice the relaxation time T1, or between the relaxation time T1 and twice the relaxation time T1.

Referring now to FIG. 17, a flow image generation process 1000 is depicted. The process 1000 can be employed with the various MRI systems and secondary pre-polarizing magnets disclosed herein. For example, the process 1000 can be utilized with the head-only MRI systems 700 (FIGS. 9-10 and 14) and 800 (FIGS. 11-13 and 15). As further described herein, the pulse sequence diagrams 902 and 904 (FIG. 16) can be utilized during the process 1000 to generate the first and second MR images. In various instances, the process 1000 can be utilized intraoperatively to obtain real-time images during a surgical procedure, such as a surgical procedure involving a robotic system, such as the robotic system 680, for example.

At the outset, block 1002, a patient is positioned on a surgical table and the area to be imaged, e.g. the brain, is positioned within the primary permanent magnet array, e.g. a domed housing. At block 1004, MR images are generated intraoperatively using a low-field magnetic field projected into the region of interest, e.g. within the domed housing. The intraoperative images can be utilized in an image-guided procedure, which can be a robotic surgical procedure and/or a non-robotic surgical procedure. In various instances, the process 1000 is further configured to utilize the process 570 (FIG. 7) to generate the various images thereof.

At block 1006, the secondary magnet(s) are moved into position. As depicted in FIGS. 9-15, the secondary magnet(s) can be positioned over a subject's upper body, or chest region, for example. At block 1008, a first image is acquired using a first polarity, e.g. a positive polarity Bₚ, for the secondary magnet(s). The pulse sequence 902 (FIG. 16) can be utilized to acquire the first image. For the first image, pre-polarization is produced in one direction, e.g., clockwise in a pre-polarizing coil, to produce arterial blood magnetization (M_{z}) in the upper body having a direction parallel to the static magnetization of stationary tissues in the subject's head. The parallel direction of M_{z} can be considered the control condition, for example, and the resulting image, i.e. the first image, can be considered the control image, for example.

At block 1010, a second image is acquired using a second polarity, e.g. a negative polarity −Bₚ for the secondary magnet(s). The pulse sequence 904 (FIG. 16) can be utilized to acquire the second image. For the second image, the pre-polarizing field direction is reversed, e.g., counterclockwise in the pre-polarizing coil, to produce arterial blood magnetization (−M_{z}) having a direction anti-parallel, or inverted, relative to the static magnetization of stationary tissues in the subject's head. The anti-parallel direction of −Bₚ for the secondary magnet(s) can be considered the labeling condition, for example, and the resulting image recorded with the −M_{z} preparation can be considered to be the tagged image, for example.

Intermediate blocks 1008 and 1010, the polarity of the secondary magnet(s) can be reversed. For example, for an electromagnet, the current direction can be reversed, and for a permanent magnet array, the magnet array can be mechanically flipped or rotated.

Acquisition of the first image may take a few minutes and acquisition of the second image may take a few minutes; however, the patient's anatomy is generally assumed to be constant over that time frame.

In various instances, acquisition of each image can involve repeatedly creating the polarization with the secondary magnet and using time-of-flight to tag the blood flowing into the region of interest.

At block 1012, the first image (obtained at block 1008) is subtracted from the second image (obtained at block 1010) to leave an image representing only the differentially pre-polarized blood signal. In other words, static signals in the region of interest, such as static portions in the brain, are subtracted out to produce the flow image.

The acquisition of a control image in step 1008 and a tagged image in step 1010 can be repeated as often as necessary to produce a desired signal level via signal averaging.

After the first image(s) and the second image(s) are acquired, the secondary magnet(s) can be removed from their position relative to the patient. For example, the secondary magnet(s) can be removed for their placement over the patient's chest.

Sequence timing comprises the duration of the pre-polarizing period, or labeling delay (LD), and the delay between the end of the pre-polarizing period and the start of brain image acquisition, or post-labeling delay (PLD). The sequence timing determines the magnitude of +/−M_{z} and the location in the vasculature where maximum signal-to-noise (SNR) and vessel visibility will be achieved, i.e., whether an angiogram or a CBF map is produced. In this way, the method 1000 relies on a dedicated +M_{z} or −M_{z} for arterial blood and is distinguishable from the use of a tuned radio-frequency coil to manipulate pre-existing polarization on blood.

In conventional MR angiography or cerebral blood flow (CBF) mapping in whole-body scanners, magnetization inversion preparation can be achieved very quickly using a small RF coil, as discussed above. Inversion of arterial blood water is produced by considering the flow velocity through the RF coil's B₁⁺ field. However, arterial blood is outside the polarizing field of a head-only MRI system. Consequently, multiples of T₁(Bₚ) must elapse in order for the magnetization to build up in the presence of the temporary pre-polarizing magnetic field. Generally, the arterial blood must experience the magnetic field of the pre-polarizing coil for 1.5 seconds or more before significant +/−M_{z} can be attained. The pre-polarizing coil must be sufficiently large to maintain the polarizing field, Bₚ, for the arterial blood as it moves from the heart towards the head in this time frame. A pre-polarizing coil that covers the heart and upper chest region should be sufficiently large to achieve high +/−M_{z} for arterial blood. Referring again to the secondary magnets 720, 722, 724, 726, 820, 822, 824, 826, the magnet(s) must be large enough to achieve the +/−M_{z} for arterial blood flowing toward the domed housing 702, 802 of the MRI system.

Another consideration is the lifetime of the arterial blood magnetization once it has been created. The Bₚ field strength can be any value, but in general the larger Bₚ the greater +/−M_{z} and the higher the final subtracted image SNR will be.

In various instances, Bₚ may not equal B₀. In fact, Bₚ>B₀ would yield an SNR benefit in various instances. Whatever the strength of Bₚ, the arterial blood magnetization is maintained in a relatively high magnetic field between the labeling position and the neck, whereupon the magnetization will begin to precess about the fringe field of the head-only magnet. For typical blood flow velocities of tens of cm per second, for MR precession frequencies generally measured in MHz, and assuming the stray fields of the pre-polarizing and main magnetic fields intersect, the arterial blood magnetization produced by the pre-polarizing coil will move along the effective field between Bₚ and B₀ adiabatically. The arterial blood magnetization will relax with its instantaneous T₁ at all points along its path. Provided the lowest field point between Bₚ and B₀ is several millitesla (hundreds of kHz Larmor frequency), and given the relatively fast passage of arterial blood, there will be only modest loss of arterial M_{z} during transit.

The magnitude of Bₚ that can be achieved is limited by several factors, including the ability to cool the pre-polarizing coil (for an electromagnet), and the degree of interaction between the pre-polarizing coil and the head-only imaging magnet. For a high duty cycle, forced air or liquid cooling may be incorporated to cool the secondary coil, for example. Safety considerations also dictate constraints on the mass and movability of the Bₚ coil, and on the switching rate (dBₚ/dt) that can be used without risk of nerve stimulation. For example, if the Bₚ coil is located such that its field overlays the heart, stringent limits of dBₚ/dt would be required. However, restricting the switching rate to safe limits would have a very small effect on the overall efficiency of the flow imaging technique because the labeling duration is long (seconds) compared to the switching time (milliseconds).

In various instances, the magnet(s) that are used to pre-polarize arterial blood outside the head might include secondary magnets designed to preserve arterial blood magnetization as it flows toward the brain. Additionally, the secondary magnet(s) might be optimized to preserve the magnetic field homogeneity across the brain, i.e. to act as local shim magnets. Overall, the effective magnetic field of the collection of magnets should satisfy the twin goals of polarizing the arterial blood and permitting imaging of the vasculature in the brain.

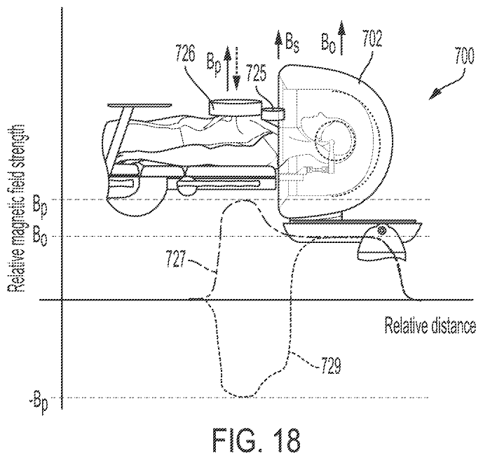

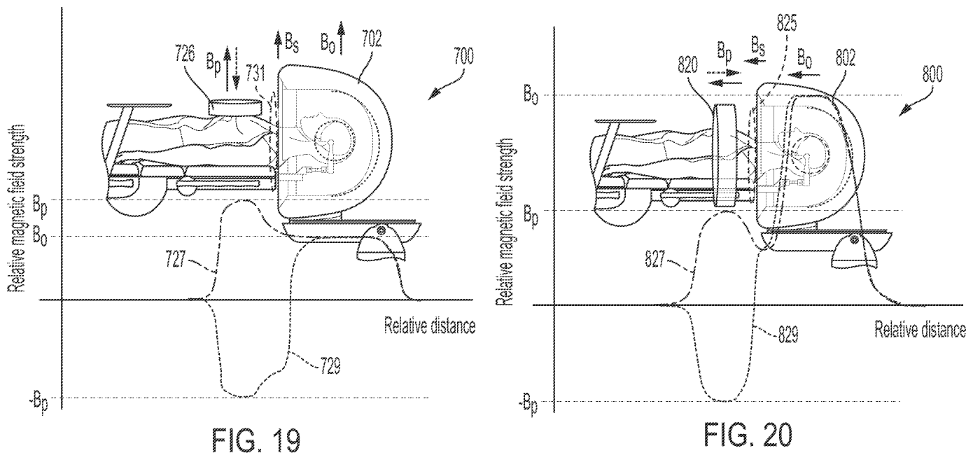

Referring now to FIGS. 18-20, the relative field strength of a pre-polarizing magnetic field and a shim magnetic field for various head-optimized MRI systems are depicted. FIGS. 18 and 19 depict examples for arterial blood labeling and control imaging conditions when the imaging magnetic field direction is orthogonal to the patient's body, i.e. anterior-posterior relative to the patient's head and along a vertical axis as oriented in FIGS. 18 and 19, are contemplated. FIG. 20 depicts an alternative example when the imaging magnetic field direction is oriented axially along the patient's body, i.e. head-to-foot relative to the patient. The pre-polarizing magnet(s) and secondary shim magnet(s) in FIGS. 18-20 can be either permanent magnets or electromagnets to produce the desired conditions for image acquisition.

Referring primarily to FIG. 18, magnetic field strengths 727, 729 are depicted for an exemplary imaging system that is similar in many aspects to the MRI system 700 (FIG. 14) and further includes a secondary magnet, or shim magnet, 725. The effective magnetic field 729 corresponds to the primary pre-polarizing magnet 726 being active for labeling and the effective magnetic field 727 corresponds to the primary pre-polarizing magnet 726 being active for control image acquisitions. The shim magnet 725, which may be a permanent magnet or an electromagnet, produces a shim field Bₛ that can be matched to the state of the primary magnet. The shim field Bₛ may be different between labeling and control image acquisition.

FIG. 18 is one example based on B₀ being oriented vertically, orthogonal to the patient body axis. The effective magnetic fields in FIG. 18 reflect a particular magnetic field control. More specifically, in the labeling condition, the effective magnetic field 729 for labeling of arterial blood is inverted, e.g. antiparallel, to −Bₚ and the shim coil magnetic field Bₛ preserves the arterial blood magnetization and maintains magnetic field homogeneity across the brain. In the control condition, the effective magnetic field 727 is a result of the primary pre-polarizing coil 726 being parallel to B₀ by sending the current in the appropriate direction (if an electromagnet) or by orienting the magnet (if a permanent magnet) to be parallel to the main magnetic field. In still other instances, a pre-polarizing magnetic field parallel to B₀ can be used for labeling and the anti-parallel direction magnetic field −Bₚ can be used for control imaging. In either case, the shim field Bₛ can be changed to maintain the desired characteristics of the effective magnetic field during the labeling control imaging conditions, i.e. maintenance of pre-polarization of arterial blood and a homogeneous magnetic field across the brain for imaging.

Referring now to FIG. 19, the MRI system 700 is depicted with a shim magnet 731. The shim magnet 731 is a circular array of permanent magnets that have the effect of extending the magnetic field of the head-only scanner towards the subject's chest. In various instances, control imaging is performed with a pre-polarizing magnetic field parallel to the main magnetic field for imaging, corresponding to the effective magnetic field 727, and labeling is performed with an anti-parallel pre-polarizing field, corresponding to the effective magnetic field 729. In still other instances, a reversed pre-polarizing magnetic field can be used for labeling and the opposite direction magnetic field can be used for control imaging.

Referring now to FIG. 20, magnetic field strengths 827, 829 are depicted for an exemplary imaging system that is similar in many aspects to the MRI system 800 (FIG. 11), including the orientation of the main magnetic field being oriented axially, along the patient's body. The MRI system 800 further includes a secondary magnet, or shim magnet, 825. In various instances, control imaging is performed with a pre-polarizing magnetic field parallel to the main magnetic field, corresponding to the effective magnetic field 827, and labeling is performed with a pre-polarizing field anti-parallel to the main magnetic field, corresponding to the effective magnetic field 829. In still other instances, a reversed pre-polarizing magnetic field can be used for labeling and the opposite direction magnetic field can be used for control imaging.

The various secondary magnets, or shim coils, disclosed herein can be selectively activated to optimize the MRI system for different uses.

### Examples

Various additional aspects of the subject matter described herein are set out in the following numbered examples:

Example 1: A method, comprising: projecting a primary static magnetic field in a field of view, wherein the primary static magnetic field comprises a low-field strength magnetic field, and wherein the field of view is defined within a head-optimized housing for magnetic resonance (MR) imaging; projecting a secondary static magnetic field to pre-polarize a first fluid outside the field of view and moving toward the field of view; transmitting a first radio frequency (RF) pulse sequence to an RF coil assembly to excite magnetization in the first fluid in the field of view; acquiring a first image of the first fluid in the field of view based on the first RF pulse sequence; projecting a tertiary static magnetic field to pre-polarize a second fluid outside the field of view and moving toward the field of view, wherein the tertiary static magnetic field is oriented opposite to the secondary static magnetic field; transmitting a second RF pulse sequence to the RF coil assembly to excite magnetization in the second fluid in the field of view; acquiring a second image of the second fluid in the field of view based on the second RF pulse sequence; and subtracting the first image from the second image to generate a flow image of an object of interest positioned in the field of view.

Example 2: The method of Example 1, further comprising positioning a secondary magnet outside of the head-optimized housing and spaced apart from the head-optimized housing, wherein the secondary magnet is configured to project the secondary static magnetic field and the tertiary static magnetic field.

Example 3: The method of claim 2, wherein the secondary magnet comprises an array of permanent magnets, and wherein the method further comprises reversing the array of permanent magnets after acquiring the first image and before projecting the tertiary static magnetic field.

Example 4: The method of any of Example 1 and 2, wherein the secondary magnet comprises a direct current polarizing coil, and wherein the method further comprises reversing the polarity of the direct current polarizing coil after acquiring the first image and before projecting the tertiary static magnetic field.

Example 5: The method of any of Examples 1-4, further comprising: transmitting, intraoperatively, a third RF pulse sequence to the RF coil assembly to excite magnetization in a human brain positioned in the field of view; acquiring, intraoperatively, an image of the human brain; and overlaying the flow image of the image of the human brain.

Example 6: The method of any of Examples 1-5, wherein the first fluid defines a relaxation time T1 at the first low-field strength, and wherein the method further comprises projecting the secondary static magnetic field for a labeling delay (LD) time period, wherein the LD time period corresponds to at least the relaxation time T1.

Example 7: The method of any of Examples 1-6, further comprising waiting a post-labeling delay (PLD) time period between projecting the secondary static magnetic field and acquiring the first image, wherein the PLD time period is less than five times the relaxation time T1.

Example 8: The method of any of Examples 1-7, wherein the primary static magnetic field defines a B₀ axis, wherein the secondary static magnetic field defines a first axis, wherein the tertiary static magnetic field defines a second axis, and wherein one of the first axis and the second axis is parallel to the B₀ axis, and wherein the other of the first axis and the second axis is antiparallel to the B₀ axis.

Example 9: The method of any of Examples 1-8, wherein projecting a primary static magnetic field in a field of view comprises projecting the low-field strength magnetic field at a field strength of less than 1 Tesla.

Example 10: The method of any of Examples 1-9, further comprising concurrently: projecting the secondary static magnetic field to pre-polarize the first fluid outside the field of view and moving toward the field of view; and transmitting the first RF pulse sequence to the RF coil assembly to excite magnetization in the first fluid in the field of view.

Example 11: A magnetic resonance (MR) imaging system, comprising: a domed housing; an array of magnets mounted to the domed housing, wherein the array of magnets is configured to generate a static, low-field strength magnetic field in a field of view, wherein the static, low-field strength magnetic field defines a B₀ axis; a radio frequency (RF) coil assembly configured to excite magnetization in an object of interest positioned in the field of view; a secondary magnet separate from the domed housing and movable between a first configuration and a second configuration, wherein the secondary magnet is configured to generate a secondary static magnetic field, and wherein the secondary static magnetic field defines a secondary axis that is parallel to the B₀ axis in the first configuration of the secondary magnetic and is anti-parallel to the B₀ axis in the second configuration of the secondary magnet; and a control circuit to: transmit RF pulse sequences to the RF coil assembly; receive first output signals from the RF coil assembly corresponding to the secondary magnet being positioned in the first configuration; receive second output signals from the RF coil assembly corresponding to the secondary magnet being positioned in the second configuration; generate an image based on the difference between the first output signals and the second output signals.

Example 12: The MR imaging system of Example 11, wherein the domed housing comprises a head-optimized housing structured and dimensioned to receive a human head, wherein the object of interest comprises a human brain, and wherein the image comprises a blood flow image of the human brain.

Example 13: The MR imaging system of Example 12, wherein the blood flow image comprises an angiogram.

Example 14: The MR imaging system of Example 12, wherein the blood flow image comprises a cerebral blood flow map.

Example 15: The MR imaging system of any of Examples 11-14, wherein the secondary magnet comprises an array of permanent magnets.

Example 16: The MR imaging system of any of Examples 11-15, further comprising a motor for flipping the secondary magnet between the first configuration and the second configuration.

Example 17: The MR imaging system of any of Examples 11-16, wherein the secondary magnet comprises a direct current polarizing coil, and wherein the control circuit is further to reverse the polarity of the direct current polarizing coil after receiving the first output signals and before receiving the second output signals.

Example 18: The MR imaging system of any of Examples 11-17, wherein the secondary static magnetic field comprises a tapered magnetic field, and wherein the tapered magnetic field tapers toward the domed housing.

Example 19: The MR imaging system of any of Examples 11-18, further comprising an array of shim magnets positioned intermediate the domed housing and the secondary magnet.

Example 20: The MR imaging system of Example 19, wherein the control circuit is further to selectively activate subsets of the array of shim magnets.

Though various aspects disclosed herein are directed to brain imaging and/or neurological interventions, the reader will appreciate that the various systems and methods disclosed herein can be used to image other portions of a patient's anatomy and/or different structures in various instances.

While several forms have been illustrated and described, it is not the intention of Applicant to restrict or limit the scope of the appended claims to such detail. Numerous modifications, variations, changes, substitutions, combinations, and equivalents to those forms may be implemented and will occur to those skilled in the art without departing from the scope of the present disclosure. Moreover, the structure of each element associated with the described forms can be alternatively described as a means for providing the function performed by the element. Also, where materials are disclosed for certain components, other materials may be used. It is therefore to be understood that the foregoing description and the appended claims are intended to cover all such modifications, combinations, and variations as falling within the scope of the disclosed forms. The appended claims are intended to cover all such modifications, variations, changes, substitutions, modifications, and equivalents.

The foregoing detailed description has set forth various forms of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, and/or examples can be implemented, individually and/or collectively, by a wide range of hardware, software, firmware, or virtually any combination thereof. Those skilled in the art will recognize that some aspects of the forms disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as one or more program products in a variety of forms, and that an illustrative form of the subject matter described herein applies regardless of the particular type of signal bearing medium used to actually carry out the distribution.

Instructions used to program logic to perform various disclosed aspects can be stored within a memory in the system, such as dynamic random access memory (DRAM), cache, flash memory, or other storage. Furthermore, the instructions can be distributed via a network or by way of other computer readable media. Thus a machine-readable medium may include any mechanism for storing or transmitting information in a form readable by a machine (e.g., a computer), but is not limited to, floppy diskettes, optical disks, compact disc, read-only memory (CD-ROMs), and magneto-optical disks, read-only memory (ROMs), random access memory (RAM), erasable programmable read-only memory (EPROM), electrically erasable programmable read-only memory (EEPROM), magnetic or optical cards, flash memory, or a tangible, machine-readable storage used in the transmission of information over the Internet via electrical, optical, acoustical or other forms of propagated signals (e.g., carrier waves, infrared signals, digital signals, etc.). Accordingly, the non-transitory computer-readable medium includes any type of tangible machine-readable medium suitable for storing or transmitting electronic instructions or information in a form readable by a machine (e.g., a computer).

As used in any aspect herein, the term “control circuit” may refer to, for example, hardwired circuitry, programmable circuitry (e.g., a computer processor including one or more individual instruction processing cores, processing unit, processor, microcontroller, microcontroller unit, controller, digital signal processor (DSP), programmable logic device (PLD), programmable logic array (PLA), or field programmable gate array (FPGA)), state machine circuitry, firmware that stores instructions executed by programmable circuitry, and any combination thereof. The control circuit may, collectively or individually, be embodied as circuitry that forms part of a larger system, for example, an integrated circuit (IC), an application-specific integrated circuit (ASIC), a system on-chip (SoC), desktop computers, laptop computers, tablet computers, servers, smart phones, etc. Accordingly, as used herein “control circuit” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

As used in any aspect herein, the term “logic” may refer to an app, software, firmware and/or circuitry configured to perform any of the aforementioned operations. Software may be embodied as a software package, code, instructions, instruction sets and/or data recorded on non-transitory computer readable storage medium. Firmware may be embodied as code, instructions or instruction sets and/or data that are hard-coded (e.g., nonvolatile) in memory devices.

As used in any aspect herein, the terms “component,” “system,” “module” and the like can refer to a control circuit computer-related entity, either hardware, a combination of hardware and software, software, or software in execution.

As used in any aspect herein, an “algorithm” refers to a self-consistent sequence of steps leading to a desired result, where a “step” refers to a manipulation of physical quantities and/or logic states which may, though need not necessarily, take the form of electrical or magnetic signals capable of being stored, transferred, combined, compared, and otherwise manipulated. It is common usage to refer to these signals as bits, values, elements, symbols, characters, terms, numbers, or the like. These and similar terms may be associated with the appropriate physical quantities and are merely convenient labels applied to these quantities and/or states.

A network may include a packet switched network. The communication devices may be capable of communicating with each other using a selected packet switched network communications protocol. One example communications protocol may include an Ethernet communications protocol which may be capable permitting communication using a Transmission Control Protocol/Internet Protocol (TCP/IP). The Ethernet protocol may comply or be compatible with the Ethernet standard published by the Institute of Electrical and Electronics Engineers (IEEE) titled “IEEE 802.3 Standard”, published in December 2008 and/or later versions of this standard. Alternatively or additionally, the communication devices may be capable of communicating with each other using an X.25 communications protocol. The X.25 communications protocol may comply or be compatible with a standard promulgated by the International Telecommunication Union-Telecommunication Standardization Sector (ITU-T). Alternatively or additionally, the communication devices may be capable of communicating with each other using a frame relay communications protocol. The frame relay communications protocol may comply or be compatible with a standard promulgated by Consultative Committee for International Telegraph and Telephone (CCITT) and/or the American National Standards Institute (ANSI). Alternatively or additionally, the transceivers may be capable of communicating with each other using an Asynchronous Transfer Mode (ATM) communications protocol. The ATM communications protocol may comply or be compatible with an ATM standard published by the ATM Forum titled “ATM-MPLS Network Interworking 2.0” published August 2001, and/or later versions of this standard. Of course, different and/or after-developed connection-oriented network communication protocols are equally contemplated herein.

Unless specifically stated otherwise as apparent from the foregoing disclosure, it is appreciated that, throughout the foregoing disclosure, discussions using terms such as “processing,” “computing,” “calculating,” “determining,” “displaying,” or the like, refer to the action and processes of a computer system, or similar electronic computing device, that manipulates and transforms data represented as physical (electronic) quantities within the computer system's registers and memories into other data similarly represented as physical quantities within the computer system memories or registers or other such information storage, transmission or display devices.

One or more components may be referred to herein as “configured to,” “configurable to,” “operable/operative to,” “adapted/adaptable,” “able to,” “conformable/conformed to,” etc. Those skilled in the art will recognize that “configured to” can generally encompass active-state components and/or inactive-state components and/or standby-state components, unless context requires otherwise.

Those skilled in the art will recognize that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to claims containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations.

In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that typically a disjunctive word and/or phrase presenting two or more alternative terms, whether in the description, claims, or drawings, should be understood to contemplate the possibilities of including one of the terms, either of the terms, or both terms unless context dictates otherwise. For example, the phrase “A or B” will be typically understood to include the possibilities of “A” or “B” or “A and B.”

With respect to the appended claims, those skilled in the art will appreciate that recited operations therein may generally be performed in any order. Also, although various operational flow diagrams are presented in a sequence(s), it should be understood that the various operations may be performed in other orders than those which are illustrated, or may be performed concurrently. Examples of such alternate orderings may include overlapping, interleaved, interrupted, reordered, incremental, preparatory, supplemental, simultaneous, reverse, or other variant orderings, unless context dictates otherwise. Furthermore, terms like “responsive to,” “related to,” or other past-tense adjectives are generally not intended to exclude such variants, unless context dictates otherwise.

It is worthy to note that any reference to “one aspect,” “an aspect,” “an exemplification,” “one exemplification,” and the like means that a particular feature, structure, or characteristic described in connection with the aspect is included in at least one aspect. Thus, appearances of the phrases “in one aspect,” “in an aspect,” “in an exemplification,” and “in one exemplification” in various places throughout the specification are not necessarily all referring to the same aspect. Furthermore, the particular features, structures or characteristics may be combined in any suitable manner in one or more aspects.

Any patent application, patent, non-patent publication, or other disclosure material referred to in this specification and/or listed in any Application Data Sheet is incorporated by reference herein, to the extent that the incorporated materials is not inconsistent herewith. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

In summary, numerous benefits have been described which result from employing the concepts described herein. The foregoing description of the one or more forms has been presented for purposes of illustration and description. It is not intended to be exhaustive or limiting to the precise form disclosed. Modifications or variations are possible in light of the above teachings. The one or more forms were chosen and described in order to illustrate principles and practical application to thereby enable one of ordinary skill in the art to utilize the various forms and with various modifications as are suited to the particular use contemplated. It is intended that the claims submitted herewith define the overall scope.

What is claimed is:

1. A method, comprising:
projecting a primary static magnetic field in a field of view, wherein the primary static magnetic field comprises a low-field strength magnetic field, and wherein the field of view is defined within a head-optimized housing for magnetic resonance (MR) imaging;
projecting a secondary static magnetic field to pre-polarize a first fluid outside the field of view and moving toward the field of view;
transmitting a first radio frequency (RF) pulse sequence to an RF coil assembly to excite magnetization in the first fluid in the field of view;
acquiring a first image of the first fluid in the field of view based on the first RF pulse sequence;
projecting a tertiary static magnetic field to pre-polarize a second fluid outside the field of view and moving toward the field of view, wherein the tertiary static magnetic field is oriented opposite to the secondary static magnetic field;
transmitting a second RF pulse sequence to the RF coil assembly to excite magnetization in the second fluid in the field of view;
acquiring a second image of the second fluid in the field of view based on the second RF pulse sequence; and
subtracting the first image from the second image to generate a flow image of an object of interest positioned in the field of view.

2. The method of claim 1, further comprising positioning a secondary magnet outside of the head-optimized housing and spaced apart from the head-optimized housing, wherein the secondary magnet is configured to project the secondary static magnetic field and the tertiary static magnetic field.

3. The method of claim 2, wherein the secondary magnet comprises an array of permanent magnets, and wherein the method further comprises reversing the array of permanent magnets after acquiring the first image and before projecting the tertiary static magnetic field.

4. The method of claim 1, wherein the secondary magnet comprises a direct current polarizing coil, and wherein the method further comprises reversing the polarity of the direct current polarizing coil after acquiring the first image and before projecting the tertiary static magnetic field.

5. The method of claim 1, further comprising:
transmitting, intraoperatively, a third RF pulse sequence to the RF coil assembly to excite magnetization in a human brain positioned in the field of view;
acquiring, intraoperatively, an image of the human brain; and
overlaying the flow image of the image of the human brain.

6. The method of claim 1, wherein the first fluid defines a relaxation time T1 at the first low-field strength, and wherein the method further comprises projecting the secondary static magnetic field for a labeling delay (LD) time period, wherein the LD time period corresponds to at least the relaxation time T1.

7. The method of claim 6, further comprising waiting a post-labeling delay (PLD) time period between projecting the secondary static magnetic field and acquiring the first image, wherein the PLD time period is less than five times the relaxation time T1.

8. The method of claim 1, wherein the primary static magnetic field defines a B₀ axis, wherein the secondary static magnetic field defines a first axis, wherein the tertiary static magnetic field defines a second axis, and wherein one of the first axis and the second axis is parallel to the B₀ axis, and wherein the other of the first axis and the second axis is antiparallel to the B₀ axis.

9. The method of claim 1, wherein projecting a primary static magnetic field in a field of view comprises projecting the low-field strength magnetic field at a field strength of less than 1 Tesla.

10. The method of claim 1, further comprising concurrently:
projecting the secondary static magnetic field to pre-polarize the first fluid outside the field of view and moving toward the field of view; and
transmitting the first RF pulse sequence to the RF coil assembly to excite magnetization in the first fluid in the field of view.

11. A magnetic resonance (MR) imaging system, comprising:
a domed housing;
an array of magnets mounted to the domed housing, wherein the array of magnets is configured to generate a static, low-field strength magnetic field in a field of view, wherein the static, low-field strength magnetic field defines a B₀ axis;
a radio frequency (RF) coil assembly configured to excite magnetization in an object of interest positioned in the field of view;
a secondary magnet separate from the domed housing and movable between a first configuration and a second configuration, wherein the secondary magnet is configured to generate a secondary static magnetic field, and wherein the secondary static magnetic field defines a secondary axis that is parallel to the B₀ axis in the first configuration of the secondary magnetic and is anti-parallel to the B₀ axis in the second configuration of the secondary magnet; and
a control circuit to:
transmit RF pulse sequences to the RF coil assembly;
receive first output signals from the RF coil assembly corresponding to the secondary magnet being positioned in the first configuration;
receive second output signals from the RF coil assembly corresponding to the secondary magnet being positioned in the second configuration;
generate an image based on the difference between the first output signals and the second output signals.

12. The MR imaging system of claim 11, wherein the domed housing comprises a head-optimized housing structured and dimensioned to receive a human head, wherein the object of interest comprises a human brain, and wherein the image comprises a blood flow image of the human brain.

13. The MR imaging system of claim 12, wherein the blood flow image comprises an angiogram.

14. The MR imaging system of claim 12, wherein the blood flow image comprises a cerebral blood flow map.

15. The MR imaging system of claim 11, wherein the secondary magnet comprises an array of permanent magnets.

16. The MR imaging system of claim 15, further comprising a motor for flipping the secondary magnet between the first configuration and the second configuration.

17. The MR imaging system of claim 11, wherein the secondary magnet comprises a direct current polarizing coil, and wherein the control circuit is further to reverse the polarity of the direct current polarizing coil after receiving the first output signals and before receiving the second output signals.

18. The MR imaging system of claim 11, wherein the secondary static magnetic field comprises a tapered magnetic field, and wherein the tapered magnetic field tapers toward the domed housing.

19. The MR imaging system of claim 11, further comprising an array of shim magnets positioned intermediate the domed housing and the secondary magnet.

20. The MR imaging system of claim 19, wherein the control circuit is further to selectively activate subsets of the array of shim magnets.
